(19) United States
(12) Patent Application Publication
(10) Pub. No.: US 2009/0194115 A1
(43) Pub. Date: Aug. 6, 2009

(54) Apparatus for prevention and treatment of decubitus ulcers

(76) Inventor: Rafael P. Squitieri, Wilton, CT (US)

Correspondence Address: WARE FRESSOLA VAN DER SLUYS & ADOLPHSON, LLP, BRADFORD GREEN, BUILDING 5, 755 MAIN STREET, P O BOX 224, MONROE, CT, 06468, US

(21) Appl. No.: 12/322,157

(22) Filed: Jan. 29, 2009

Related U.S. Application Data
(60) Provisional application No. 61/063,273, filed on Jan. 31, 2008.

(51) Int. Cl. A61F 13/00

(52) U.S. Cl. 128/847; 128/889

(57) ABSTRACT

An apparatus to prevent and/or treat pressure ulceration of the skin notably in the sacral area, but also other pressure-sensitive areas of the body as well. The apparatus includes a garment configured for placement about a region of a person, the garment having at least one bladder and at least one corresponding support surface configured to be maintained in a substantially fixed relationship relative to a portion of the region of the person and configured to provide controllable support to the person in this portion of the region. The at least one bladder is controllably inflated/deflated with a fluid. The apparatus can be configured to off-load the sacrum and reposition weight distribution in the sacral area to avoid skin ischemia and skin breakdown leading to pressure sores know as decubitus ulcers.

### CROSS REFERENCE TO RELATED APPLICATION

Reference is made to and priority claimed from U.S. provisional application Ser. No. 61/063,273 filed Jan. 31, 2008, entitled INFLATABLE/DEFLATABLE GARMENT FOR PREVENTION AND TREATMENT OF DECUBITUS ULCERS.

### BACKGROUND OF THE INVENTION

1. Technical Field

This invention relates generally to an apparatus for the prevention and treatment of decubitus (pressure) ulcers.

2. Description of Background Art

Decubitus pressure ulcers are a common and often avoidable complication in many bed bound or wheelchair bound individuals. These pressure skin ulcerations are a result of steady pressure in one location, like the sacrum or heel, most notably in patients who are in bed for prolonged periods of time. Often times these patients are older, malnourished and incontinent, all factors predisposing patients to skin break down and ulceration. These patients are often not ambulatory and sit for prolonged periods of time in the same position either in bed or in a wheelchair. These individuals often are unable to reposition themselves to alleviate the pressure. The pressure on the skin causes ischemia, or lack of blood flow to the area, and skin breakdown results. Once the ulceration has formed and the skin barrier is broken, infection may more readily enter the body causing severe infection and sepsis. The resulting infection often times leads to further disability and death.

What is needed therefore is a way to prevent skin ulceration on an immobile or relatively immobile person resulting from sustained pressure on at least one area of the body.

### SUMMARY OF THE INVENTION

The invention presented here is an apparatus including an inflatable/deflatable garment which incorporates one or more inflatable bladders (such as air bladders) into a portion of the garment (e.g., a diaper-like garment) in order to redistribute a person's weight and prevent skin breakdown or pressure ulcers. The bladders are designed to lift and roll a patient who is unable to perform this natural movement on his or her own. This movement repositions the patient and prevents pressure sores by avoiding skin tissue ischemia, a major cause of decubitus ulcers. The bladders may be either integral, i.e. incorporated into the garment, or the bladders may be removable, i.e. inserted into pockets built into the garment. The removable bladders may be reusable as they are not an integral part of the garment itself. The garment is configured to hold the bladders in the correct location beneath the patient so that support surfaces representing portions of the garment between the bladders and the patient remain fixed relative to a portion of the patient such as a body part that requires pressure relief. The bladders are connected to a fluid pressurizing pump, such as an air compressor, and a controller. The controller can deliver a predetermined volume of air (or other fluid) at various pressures as needed to achieve effective weight redistribution and pressure relief beneath the wearer. In one embodiment, the bladders are inflated and deflated in a sequential fashion to move the patient in a predetermined fashion. The air compressor can measure the air pressure within the air bladders and deliver preset air pressures as desired. The bladders are designed in shapes which maximally unload the pressure points between the patient and the surface that the patient is on. The patient may be in any position from laying flat in bed to seated fully upright as in a wheelchair, or any position in between. The bladders are connected to the compressor via a quick coupler and a flexible tubing.

As opposed to other support surfaces, such as medical air mattresses where the patient is free to move over the surface in any direction, the invention fixes the support surfaces to the patient and therefore allows direct control over where and how the garment impacts the wearer. This allows for the support surfaces to be more effective in pressure relief and also allows for much smaller support surfaces to be used, thereby offering a substantial cost savings.

In accordance with a first broad aspect of the invention, an apparatus is provided for preventing or treating ulceration of skin, comprising a garment configured for placement about a region of a person, the garment containing at least one bladder and at least one corresponding support surface configured to be maintained in a substantially fixed relationship relative to a portion of the region of the person and configured to provide controllable support to said person in at least said portion of said region, and means for allowing a fluid to enter said at least one bladder, said means configured to controllably receive said fluid so that said at least one bladder controllably inflates and deflates. In some embodiments, at least one of said at least one bladder is integrally formed in said garment. In some embodiments, the apparatus comprises a plurality of bladders, each integrally formed in said garment. In some embodiments, the garment includes at least one pocket dimensional for receipt of a separate bladder. In some embodiments, the apparatus comprises a plurality of pockets, each dimensional for receipt of a separate bladder. In some embodiments, the means for allowing fluid to enter said at least one bladder is a tube coupled to said at least one bladder. In some embodiments, the apparatus comprises at least one outlet associated with each bladder for release of fluid in said bladder. In some embodiments, the at least one outlet is positioned relative to the garment so as to provide said release of fluid in said bladder toward the skin of said person. In some embodiments, the apparatus comprises a plurality of bladders and a corresponding plurality of support surfaces, and at least one outlet associated with each bladder and corresponding support surface for release of fluid in said bladders. In some embodiments, the at least one outlet associated with each bladder and corresponding support surface is positioned so as to provide said release of fluid in said bladder toward the skin of said person. In some embodiments, said fluid has a therapeutic quality. In some embodiments, the apparatus comprises means for controlling the fluid associated with the means for allowing a fluid to enter said at least one bladder. In some embodiments, the means for controlling the fluid comprises a fluid pressurizing pump, and a controller in cooperative engagement with the fluid pressurizing pump, the controller for controlling the inflation and deflation of said at least one bladder. In some embodiments, the apparatus comprises a plurality of bladders and wherein said controller is configured to inflate and deflate said bladders in a predetermined order. In some embodiments, the apparatus comprises a plurality of bladders, wherein the controller is configured to inflate and deflate said bladders in a random manner. In some embodiments, the controller is configured to cause inflation and deflation of at least one of the at least one bladder in a random manner. In some embodiments, the fluid pressurizing pump is an air pump. In some embodiments, the means for allowing a fluid to enter said at least one bladder is a tube extending from said at least one bladder. In some embodiments, the means for allowing the fluid to enter said at least one bladder includes a quick connect coupler dimensioned for mating with the tube. In some embodiments, the means for controlling the fluid delivers a measured volume and pressure of fluid into the at least one bladder, wherein the garment has a diaper-like configuration which positions the at least one bladder beneath said person in a position and orientation so as to reduce pressure exerted on pressure points in order to prevent development of pressure sores and ulceration of skin. In some embodiments, the apparatus comprises means for affixing the garment to said region of the person. In some embodiments, said means for affixing comprise lock and loop fasteners. In some embodiments, the garment is at least partially formed from a fabric comprising said loops. In some embodiments, said means for affixing comprises adhesive tape. In some embodiments, the apparatus comprises at least two bladders in the form of interdigitated fingers. In some embodiments, the apparatus comprises at least three bladders positioned in the garment so as to form a circular pattern. In some embodiments, the apparatus comprises an absorbent material. In some embodiments, said absorbent material is positioned relative to the garment so as to form said at least one corresponding support surface. In some embodiments, the apparatus comprises a plurality of bladders each positioned in the garment so that inflation of any of the bladders does not constrict the garment against the person. In some embodiments, the garment comprises flaps for attaching the garment to the person and wherein the plurality of bladders are positioned in the garment away from the flaps. In some embodiments, at least one support surface is positioned so as to provide relief of pressure to the person at a desired portion of said region. In some embodiments, the apparatus comprises a plurality of bladders and corresponding plurality of support surfaces so as to impart a turning motion to said region when said bladders are inflated/deflated in a particular pattern. In some embodiments, said garment is fabricated from a material including at least one of the following: polyurethane, polyvinylchloride, polyethylene, polypropylene, cotton, biological materials, and biodegradable materials. In some embodiments, the garment is fabricated from a reusable material. In some embodiments, the garment is fabricated from a disposable material. In some embodiments, the apparatus comprises means for conditioning the fluid prior to entry in said at least one bladder. In some embodiments, said conditioning is at least heating and/or cooling said fluid. In some embodiments, the apparatus further comprises a dressing. In some embodiments, said dressing is configured to aid in the treatment or prevention of skin ulceration. In some embodiments, said at least one bladder and corresponding support surface is configured to provide relief of pressure associated with a bony prominence of said person.

In accordance with a second broad aspect of the invention, a system is provided for preventing or treating ulceration of skin, comprising a garment configured for placement about a region of a person, the garment containing a plurality of bladders and a corresponding plurality of support surfaces each configured to be maintained in a substantially fixed relationship relative to a portion of the region of the person and configured to provide controllable support to said person in at least said portion of said region, means for allowing a fluid to enter each bladder of said plurality of bladders, a fluid pressurizing pump, and a controller in cooperative engagement with the fluid pressurizing pump, the controller configured to control the inflation and deflation of each of the plurality of bladders.

In accordance with a third broad aspect of the invention, a method for preventing or treating ulceration of skin is provided, comprising placing a garment about a region of a person, the garment containing a plurality of bladders and a corresponding plurality of support surfaces each configured to be maintained in a substantially fixed relationship relative to a portion of the region of the person and configured to provide controllable support to said person in at least said portion of said region, and controllably inflating and deflating each bladder of said plurality of bladders. In some embodiments, at least some of the fluid exits each of the bladders of said plurality of bladders during deflation thereof directed toward the skin of said person. In some embodiments, the inflating and deflating of each bladder of said plurality of bladders is in a predetermined order. In some embodiments, the inflating and deflating of each bladder of said plurality of bladders is performed in a random manner.

### BRIEF DESCRIPTION OF THE DRAWINGS

Other features and benefits of the invention will become readily apparent from the following written description of exemplary embodiments taken in conjunction with the drawing figures wherein:

FIG. 1—A plan view of one embodiment of an apparatus including an inflatable/deflatable garment showing two air bladders manufactured into the seat portion of a diaper-like garment designed to position the air bladders beneath the sacrum to off-load pressure points underneath a person at rest in a bed or chair.

FIG. 2—A cross-sectional view taken along line 2-2 of FIG. 1, which shows the absorbent pad of the diaper-like garment and air bladders.

FIG. 3—An overview of an apparatus including an inflatable/deflatable garment connected to an air compressor controller via a flexible tubing.

FIG. 4—A perspective view of an apparatus including an inflatable/deflatable garment viewed from the back with the garment in a closed configuration showing a location of the air bladders.

FIG. 5—A perspective view of an apparatus including an inflatable/deflatable garment with removable air bladders which may be fitted into pockets in the back side of the garment to position the air bladders and hold them in the correct location beneath the wearer.

FIG. 6—A plan view of another embodiment of an apparatus including an inflatable/deflatable garment showing two air bladders arranged as interdigitated fingers and bladders manufactured into the seat portion of a diaper-like garment.

FIG. 7—A plan view of another arrangement of the two air bladders of the garment arranged as interdigitated fingers.

FIG. 8—A plan view of yet another embodiment of an apparatus including an inflatable/deflatable garment showing three air bladders arranged in a somewhat circular pattern and manufactured into the seat portion of a diaper-like garment.

FIG. 9—A plan view of the three air bladders of FIG. 8.

FIG. 10—A perspective view of still another embodiment of an apparatus including an inflatable/deflatable garment showing the inflatable/deflatable garment stitched to the backside of an absorbent diaper.

FIG. 11—A plan view of another embodiment of an apparatus including an inflatable/deflatable garment showing two air bladders arranged as interdigitated fingers, wherein the inflatable/deflatable garment includes three flaps or wings stitched or otherwise adhered to the inflatable/deflatable garment.

FIGS. 12a and 12b—A plan and perspective view of an absorbent diaper having an adhesive portion located on the front flap for engaging with the inflatable/deflatable garment of FIG. 11.

FIG. 13—A perspective view of an apparatus including an inflatable/deflatable garment and absorbent diaper of FIGS. 11 and 12 showing the inflatable/deflatable garment wrapped around and coupled to the absorbent diaper.

FIGS. 14a, 14b and 14c—Perspective views of yet another embodiment of the invention showing an apparatus including an inflatable/deflatable garment having one air bladder manufactured into the heel portion of a sock-like garment designed to position the air bladders beneath the heel to off-load pressure points underneath the heel of a person at rest in a bed.

FIG. 15 is a flow chart illustrating a method for preventing or treating ulcerations of skin, according to an embodiment of the invention.

### DETAILED DESCRIPTION

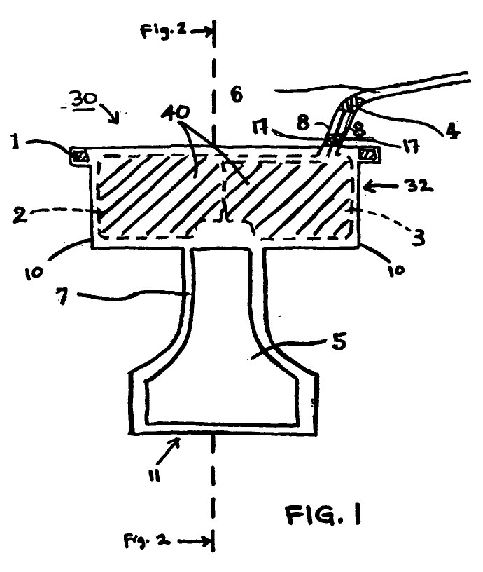

Referring now to FIG. 1, therein illustrated is plan view of one embodiment of an apparatus 30 including an inflatable/deflatable garment 32 showing bladders (such as an air bladder) manufactured into a seat portion of a diaper-like garment designed to position the air bladders beneath the sacrum to off-load pressure points underneath a person at rest in a bed or chair. The garment is comprised of an impervious outer layer 7, in the general shape of a diaper, i.e. somewhat T-shaped, having a vertical portion (creating the front flap 11) and a horizontal portion (creating the rear and side flaps 10). The garment comprises an absorbent pad 5, located in the front, or vertical, portion, and two bladders 2, 3, (e.g. air bladders) located in the rear, or horizontal, portion (shown in phantom). Flexible tube 6 connects to air bladders 2, 3 via a quick coupler 4 associated with tubes 8 connected to bladders 2 and 3. The portions of the garment on the opposite side between the bladders and the wearer (patient) form support surfaces 40 that are fixed relative to the wearer as explained more fully below.

Two self-adhesive or hook-and-loop type (e.g. Velcro™) fasteners 1 are affixed to the impervious outer layer 7, located on the outermost borders of the side flaps 10. If adhesive “tape-style” fasteners are used, a smooth non-fabric material should be used to form at least the portion of the garment that receives the tape. If a hook-and-loop type fastener is used, the opposing adhering material (i.e. either the “hook” or the “loop” material) should be affixed to the front portion of the garment in order for the hooks and loops to interweave with one another for a secure connection. The loop fasteners may be formed in at least a portion of the fabric used to make the garment. Although the embodiments of the invention described herein utilize adhesive or hook-and-loop means, the scope of the invention is not intended to be limited to any particular fastener now known or developed in the future.

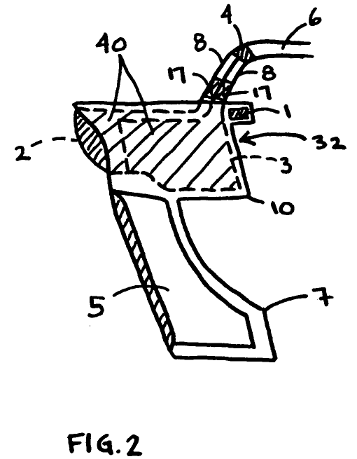

As shown in the cross-sectional view of the garment in FIG. 2, when inflated, a pocket of air expands (inflates) each cavity within the air bladders 2, 3.

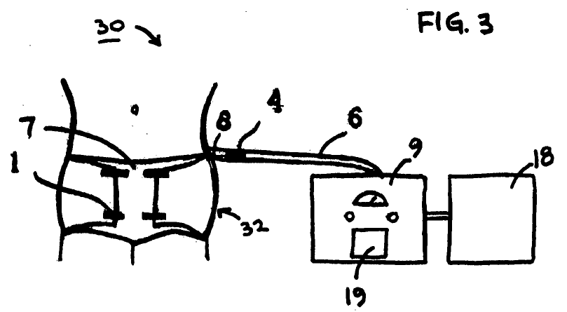

FIG. 3 reveals a front-view of the garment as fitted over a region of a patient (typically a person, but possibly a non-human animal). When fitted on the patient, absorbent pad 5 is in contact with the skin of the patient from the area below the navel to the groin. Fasteners 1 are affixed to the impervious outer layer 7 on the front portion of the garment directly opposite the absorbent pad 5, thereby creating two side flaps 10 wrapping around the patient's hips. As shown, the flexible tubing 6 is connected to a controller 9 (e.g. an air compressor controller), which provides air to the bladders 2, 3 via the flexible tubing 6 and quick coupler 4. The portion of the garment positioned between each bladder and the person thereby forms a support surface 40 (see FIGS. 1, 2, 4, and 5) which controllably applies and removes support to this portion of the region as the corresponding bladder is inflated/deflated. Each support surface has a substantially fixed spatial relationship relative to portions of the patient that the support surface is adjacent to. This fixed spatial relationship between the support surfaces and the patient provides important advantages to the patient since the support surfaces can be controlled to provide and release pressure (support) to specific portions of the patient (i.e., to the skin of the patient at fixed positions) via controlling the inflation and deflation of the corresponding bladders of the apparatus.

For all of the embodiments of the present invention, the support surfaces are the areas of the garment between a bladder and the wearer of the apparatus. If an absorbent material is added to the apparatus, the support surface may be an area of the absorbent material between the wearer and the bladder.

Although the embodiments of the invention described herein generally describe the use of flexible tubing and couplers to connect the bladders to the compressor, other connecting means may be used as well; thus the scope of the invention is not intended to be limited to any particular connector now known or developed in the future.

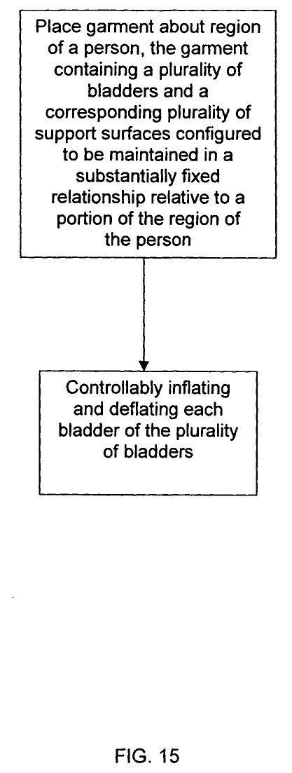

The order of inflation/deflation of bladders 2, 3 can be controlled by controller 9 (such as an air compressor controller) so as to generate a desired inflation/deflation pattern for particular patient applications. Such control can be effected by selectively controlling opening and closing of valves 7 by controller 9, or by using a plurality of tubes 6, 8 each connected to one or more bladders 2, 3. Fluid, such as air, is provided to the controller 9 by fluid pressurizing pump 18, which may be coupled to controller 9 as a separate unit or may be integral with controller 9 housed in one unit. Controller 9 can include a processor under program control (not shown) for performing such controlled inflation/deflation of bladders 2, 3. Controller 9 may further include a conditioning unit 19 for heating and/or cooling and/or humidifying the air flowing into the bladders. FIG. 15 illustrates a methodology of an embodiment of the invention. The scope of the invention is not intended to be limited to any particular pressurizing device, controller device or heating/cooling device now known or developed in the future.

In any of the embodiments disclosed herein, the maximum pressure used in inflating the bladders may range between 20-80 mmHg. Typical time intervals may be between 1-5 minutes. In one example, air bladders 2, 3 inflate to the maximum pressure for 2 minutes; then air bladder 2 deflates while air bladder 3 remains inflated for 5 minutes; then air bladder 2 reinflates and air bladders 2, 3 remain inflated for 2 minutes; then air bladder 3 deflates and air bladder 2 remains inflated for 5 minutes; then air bladders 2, 3 each partially deflate for 2 minutes; and finally the cycle repeats. Thus, the timing of the inflating of the bladders may vary to maximize the ability to relieve pressure on the wearer. The scope of the invention is not intended to be limited to any particular pressure or cycle of inflation/deflation now known or developed in the future.

Although the embodiments of the invention described herein generally describe the use of air to inflate the bladders, other fluids such as gel or liquid may be used as well; thus the scope of the invention is not intended to be limited to any particular fluid now known or developed in the future.

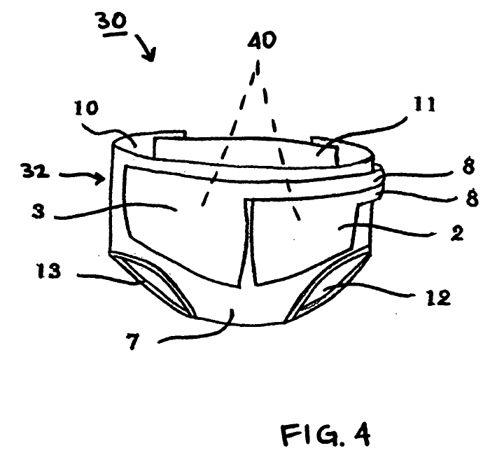

FIG. 4 shows a detailed perspective back view of the garment of FIG. 1. As presented, all elements of the garment are affixed to impervious outer layer 7 thereby insuring a fixed relationship of the support surfaces to the wearer of the apparatus. Air bladders 2, 3 are connected to air intake/egress tubes 8, 8 and are affixed to the rear portion of the garment. Flaps 10 from the rear portion of the garment are wrapped around and affixed to flap 11 of the front portion of the garment. This configuration creates two openings 12 for the lower extremities having cuffs 13.

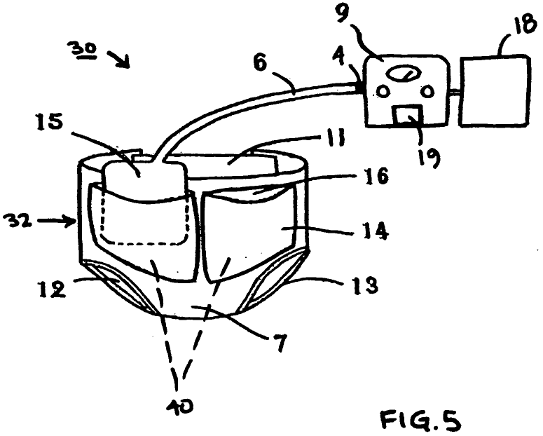

FIG. 5 shows a detailed perspective back view of the garment in an alternative embodiment having removable bladders. As shown, removable bladder 15 (e.g., an air bladder) is inserted into a pocket 14 dimensioned for the bladder through an opening 16 in the pocket 14. The air bladder 15 is continuous with flexible air tubing 6, which is coupled to controller 9 via coupler 4. Aside from the removable bladders 15 and corresponding pockets 14, the garment is fabricated in a substantially similar manner as the first embodiment, having an impervious outer layer 7, flaps 10, 11, openings 12 for the lower extremities having cuffs 13, and sets of fasteners 1 for fastening together flaps 10 and 11.

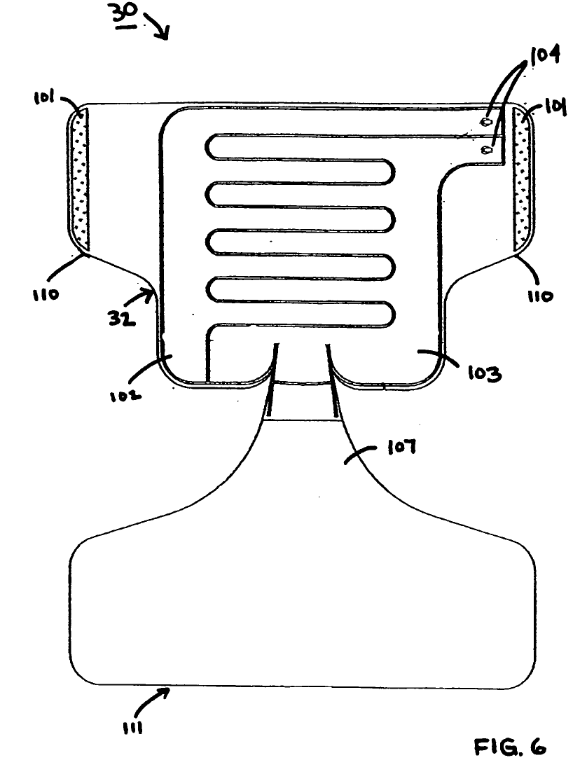

FIG. 6 shows a plan view of an alternative embodiment of the inflatable/deflatable garment showing two air bladders arranged as interdigitated fingers and the air bladders manufactured into the seat portion of a diaper-like garment. Similar to the arrangement of FIGS. 1-4, the garment is comprised of an impervious outer layer 107, in the general shape of a diaper having a front flap 111 and a rear portion having side flaps 110. Two self-adhesive or hook-and-loop type (e.g. Velcro™) fasteners 101 are affixed to the impervious outer layer 107, and located on the outermost borders of the side flaps 110. The garment comprises two bladders 102, 103 (e.g., air bladders), located in the rear, or horizontal, portion. Flexible tubing (not shown) is connected to bladders 102, 103 via elbows 104 and quick couplers (not shown).

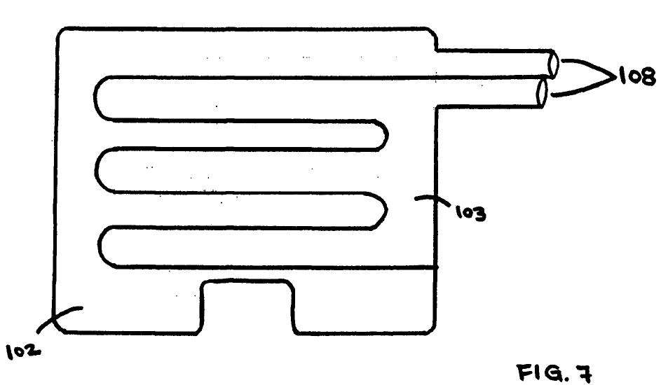

FIG. 7 shows a plan view of an alternative arrangement of the interdigitated bladders of FIG. 6. In this arrangement, bladders 102, 103 are continuous with flexible tubes 108, which are coupled to a controller (not shown).

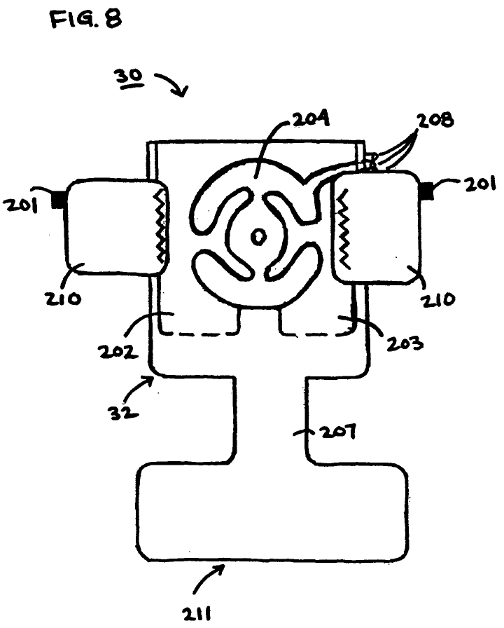

FIG. 8 shows a plan view of another embodiment of the inflatable/deflatable garment showing three air bladders arranged in a somewhat circular pattern with the air bladders manufactured into the seat portion of a diaper-like garment. Similar to the arrangement of FIGS. 1-4, the garment is comprised of an impervious outer layer 207, in the general shape of a diaper having a front flap 211 and a rear portion having side flaps 210. Different from the previous embodiments, FIG. 8 shows side flaps 210 being stitched or otherwise affixed to the inner surface of the garment and directed away from the edge of the inflatable area containing the air bladders. Two self-adhesive or hook-and-loop type (e.g. Velcro™) fasteners 201 are affixed to the outermost borders of the side flaps 210. The garment comprises three bladders 202, 203, 204 (e.g., air bladders) located in the rear portion. Flexible tubing 208 is connected to air bladders 202, 203, 204.

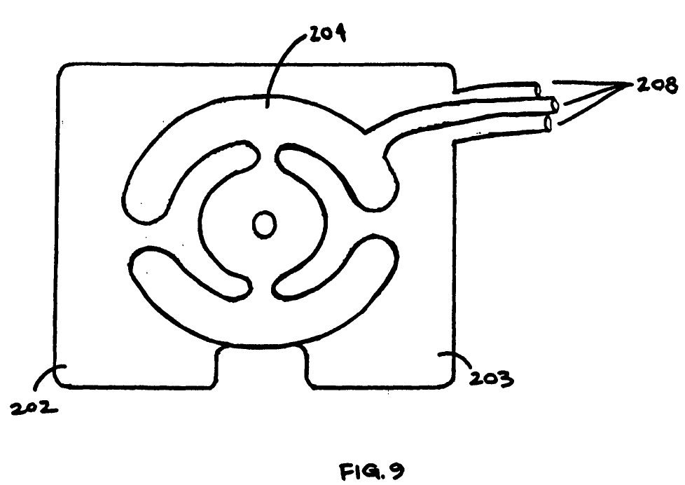

FIG. 9 shows an exploded plan view of the arrangement of the bladders (e.g. air bladders) of FIG. 8. As shown, bladders 202, 203, 204 (e.g., air bladder) are continuous with flexible air tubes 208, which are coupled to a controller (not shown). Although the connection between the air bladders and the air compressor has been described using flexible tubing continuous with the air bladders as well as flexible tubing connected to the air bladders via quick couplers, the scope of the invention is not intended to be limited to any particular type of connection between the air bladders and air compressor now known or developed in the future

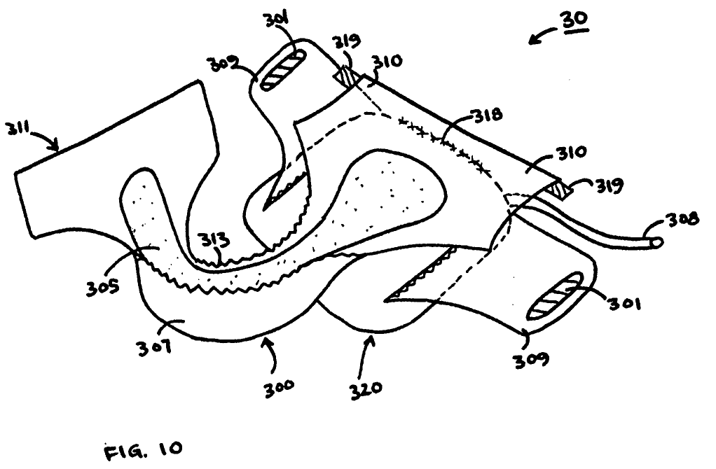

FIG. 10 shows a perspective view of another alternative embodiment of the inflatable/deflatable garment. In this arrangement, a diaper-like garment 300 is provided as well as an inflatable/deflatable portion 320. The diaper-like garment 300 and the inflatable/deflatable portion 320 are coupled together via stitching 318 or other attaching means, such as heat-sealing or crimping. Similar to the arrangement of FIGS. 1-4, the diaper-like garment 300 is comprised of an impervious outer layer 307 having a front flap 311 and a rear portion having side flaps 310). An absorbent pad 305 is affixed to the inner surface of the diaper-like garment 300. Two self-adhesive or hook-and-loop type (e.g. Velcro™) fasteners 319 are affixed to the impervious outer layer 307, located on the outermost borders of the side flaps 310. Similar to the arrangement of FIG. 8, FIG. 10 shows flaps 309 being stitched or otherwise affixed to the inner surface of the inflatable/deflatable portion 320 and directed away from the edge of the inflatable/deflatable portion 320 containing air bladders (not shown). Two self-adhesive or hook-and-loop type (e.g. Velcro™) fasteners 301 are affixed to the outermost borders of flaps 309. Though not shown in FIG. 10, the inflatable/deflatable portion 320 comprises one or more air bladders. Flexible tubing 308 is connected to the air bladders.

When worn, side flaps 310 from the rear portion of the diaper-like garment 300 are wrapped around and affixed to the front flap 311. Flaps 309 of the inflatable/deflatable portion 320 also are wrapped around and affixed to the front flap 311. This configuration creates two openings for the lower extremities having elastic cuffs 313.

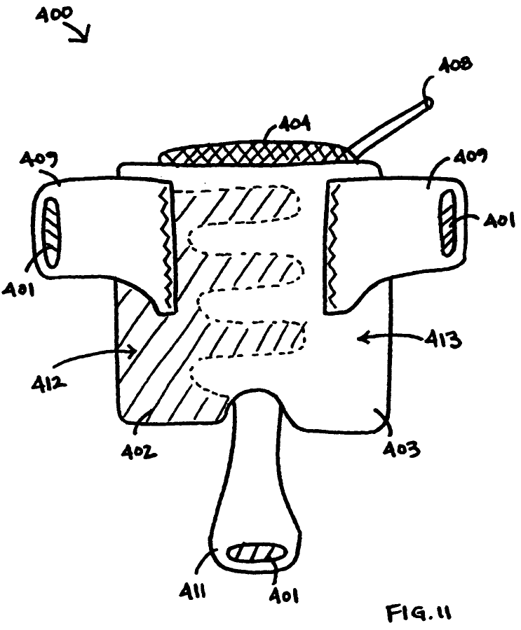

FIGS. 11-13 show yet another embodiment of the invention in which an inflatable/deflatable portion 400 and a diaper-like garment 420 are separate pieces. FIG. 11 shows the inner surface of the inflatable/deflatable portion 400, i.e. the portion facing the wearer's skin. Similar to the arrangement of FIGS. 6-7, air bladders 402, 403 are arranged as interdigitated fingers. When bladder 402 is inflated, the corresponding area of the inflatable/deflatable portion 400 facing the wearer's skin becomes support surface 412; i.e. support surface 412 supports the affected region of the body in such a manner that pressure is relieved on the affected region. In the same way, when bladder 403 is inflated, the corresponding area of the inflatable/deflatable portion 400 facing the wearer's skin becomes support surface 413; i.e. support surface 413 supports the affected region of the body in such a manner that pressure is relieved on the affected region.

This concept of the support surface may be applied to all embodiments of the invention disclosed herein. In any of these embodiments, the support surface of the apparatus is reliably fixed to the wearer and the controlled inflation/deflation of the bladders promotes a predictable and effective relief of pressure on the affected area of the body, most notable in the areas of bony prominence when the wearer is in a seated or supine position, or any position in which pressure is asserted on an area of the body. In the case of interdigitated bladders inflating and deflating in a predetermined pattern, pressure and duration, the inflated portions cause a decreased pressure on the wearer in the areas of the deflated portions thereby preventing and/or treating pressure injury. Depending on the location of the bladders and corresponding support surfaces, a rocking and/or turning motion may be applied to the wearer based on the pattern of inflation/deflation of the bladders.

Similar to the arrangement of FIGS. 8 and 10, flaps 410 are stitched or otherwise affixed to the inner surface of the inflatable/deflatable portion 400 and directed away from the edge of the inflatable/deflatable portion 400 containing bladders 402, 403 (e.g., air bladders). Different than any of the previously described embodiments, inflatable/deflatable bladder portion 400 also contains another flap 411 located perpendicular to flaps 409 and at the bottom portion of the inflatable/deflatable portion 400. Three hook-and-loop type (e.g. Velcro™) fasteners 401 are affixed to the outermost borders of flaps 409, 411. Inflatable area 404 shows the inflation of air bladders 402, 403 expanding in a direction opposite the wearer's skin. Flexible tubing 408 is connected to air bladders 402, 403.

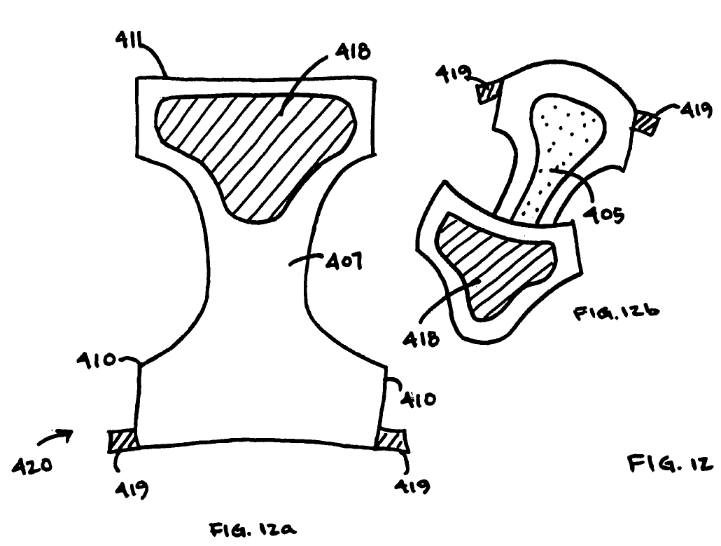

FIG. 12a shows a plan view of the diaper-like garment 420, which comprises an impervious outer layer 407 having a front flap 411 and a rear portion having side flaps 410. An absorbent pad 405 is affixed to the inner surface of the diaper-like garment 420. Two self-adhesive or hook-and-loop type (e.g. Velcro™) fasteners 419 are affixed to the impervious outer layer 407, located on the outermost borders of the side flaps 410. An adhesive area 418 comprised of hook-and-loop material is located on the front portion of the diaper-like garment 420 facing away from the wearer's skin.

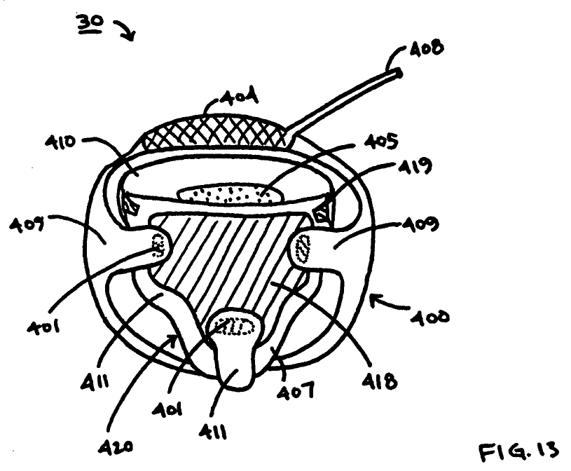

FIG. 13 shows a perspective view of the diaper-like garment 420 and inflatable/deflatable portion 400 when coupled together as worn by a patient. When fitted on the patient, absorbent pad 405 is in contact with the skin of the patient from the area below the navel to the groin. Fasteners 419 are affixed to the impervious outer layer 407 on the front portion of the garment directly opposite the absorbent pad 405, thereby creating two side flaps 410 wrapping around the patient's hips. Inflatable/deflatable portion 400 is then wrapped around the diaper-like garment 420. Flaps 409 wrap around the patient's hips and fasteners 401 adhere to adhesive area 418. Flap 411 is guided between the wearer's legs and is affixed to adhesive area 418 via fastener 401. Inflatable area 404 containing air bladders 402, 403 (not shown) expands in a direction opposite the wearer's skin. Flexible tubing 408 is coupled to air bladders 402, 403 and further coupled to an air compressor control unit (not shown), which provides air to the air bladders 402, 403 via the flexible tubing 408.

In all the previously described embodiments, the garment is constructed in such a manner that when inflated, a constricting force is not exerted on the wearer. This is because the side flaps attaching to the rear portion wrap around the front of the wearer and originate away from the leading edge of the inflating areas; i.e. the flaps are attached to the inner surface facing the wearer's skin, not on the outer surface on top of the inflating area. Thus, when the bladders are inflated, the inflating area projects away from the wearer but does not cause a shortening effect on the flaps.

In the embodiments having bladders with interdigitated fingers or intertwined sections, it has been found that pressure relief is most effective because a void is created between the inflated cells, although all the cells may be simultaneously inflated on occasion during the inflation/deflation cycle.

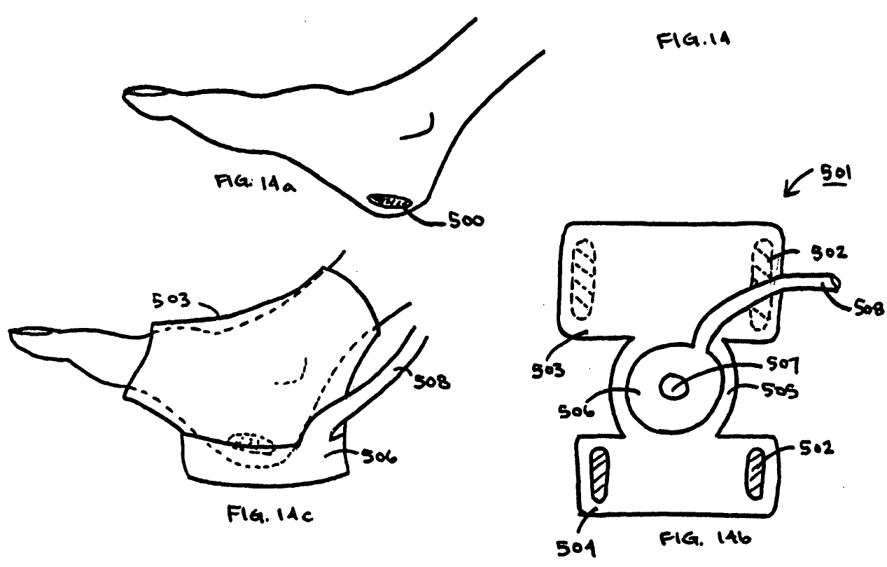

FIGS. 14a, 14b and 14c show perspective and plan views of yet another embodiment of the invention in which the apparatus and its associated inflatable/deflatable garment is utilized on another portion of the body other than the sacral area. FIG. 14a shows a heel ulcer 500. FIG. 14b shows an inflatable/deflatable apparatus comprising a garment 501 having two side portions 503, 504 and a central portion 505. Located on the outermost edges of side portions 503, 504 are self-adhesive or hook-and-loop type (e.g. Velcro™) fasteners 502. One set of fasteners (i.e., e.g. the two fasteners located on side portion 503) is located on one the surface of the garment while the other set of fasteners (i.e., e.g. the two fasteners located on side portion 504) is located on the opposite surface of the garment. The garment comprises one bladder 506 (e.g., an air bladder), which is somewhat ring-shaped having a void 507. Flexible tubing 508 is coupled to air bladder 506 and further coupled to an air compressor control unit (not shown), which provides air to the air bladder 506 via the flexible tubing 508.

As shown in FIG. 14c, when worn by a patient, the patient's heel ulcer 500 is placed in the void 507 in order to prevent any pressure being applied to the ulcer. Side portions 503, 504 are wrapped around the patent's ankle and fasteners 502 couple to one another to create a secure fit and hold the air bladder 506 in place.

Although the embodiments of the invention described herein primarily describe the use of inflatable/deflatable garment on the sacral area or heel area, other areas of the body, such as the elbows or shoulders, may also be targeted. The shape of the garment may vary to best fit the body part to be treated. The scope of the invention is not intended to be limited to any shape of the garment now known or developed in the future.

The invention as in all embodiments described herein may be entirely reusable, partially reusable or entirely disposable.

The invention as in all embodiments described herein may be further include a medicated or non-medicated wound dressing in the garment (i.e., e.g. in the disposable diaper portion) such that the dressing at least partially covers the affected area and thereby aids in the treatment and prevention of pressure ulcers.

Although some of the embodiments of the invention described herein describe the use of integral bladders while other embodiments describe the use of removable, separate bladders, it is to be understood that in any of the embodiments, the garment may be manufactured to include integral bladders or removable bladders, as needed; thus the scope of the invention is not intended to be limited to any particular manner of coupling the bladders to the garment now known or developed in the future.

In all embodiments of the invention described herein, the inflation of the bladders may effect a turning or rolling motion on the wearer, turning or rolling the wearer from side to side (i.e., e.g. if in a supine position, i.e. laying on the back facing upwards) for the purpose of rotational pressure relief beneath the wearer.

In all embodiments of the invention described herein, the garment may be manufactured from multiple natural and/or synthetic materials for maximum patient comfort, including but not limited to polyurethane, (PVC) polyvinylchloride, polyethylene, polypropylene, cotton, biological materials and/or biodegradable material; thus the scope of the invention is not intended to be limited to any particular garment material now known or developed in the future.

Furthermore, in all embodiments of the invention described herein, the garment may be designed such that the air/fluid used to inflate the air bladders may exit the garment via small openings (perforations). These perforations may control the volume of air/fluid exiting the bladders and may control the pressure within the bladders, similar to a pressure pop-off valve. By placing the perforations on the side of the bladders and on the garment material closest to the wearer's skin, the air/fluid (i.e. oxygen or medical gas) exiting the bladders may be directed toward the wearer to keep the wearer's skin dry and temperature controlled within the garment. The fluid may also contain agents that can provide an additional therapeutic effect on the wearer.

The scope of the invention is not intended to be limited to any particular shape of the air bladders now known or developed in the future.

It should be understood that, unless stated otherwise herein, any of the features, characteristics, alternatives or modifications described regarding a particular embodiment herein may also be applied, used, or incorporated with any other embodiment described herein.

The preferred embodiment described above admirably achieves the objects of the invention; however, it will be appreciated that various other additions and omissions can be made by those skilled in the art without departing from the spirit and scope of the present invention.

What is claimed is:

1. An apparatus for preventing or treating ulceration of skin, comprising:
a garment configured for placement about a region of a person, the garment containing at least one bladder and at least one corresponding support surface configured to be maintained in a substantially fixed relationship relative to a portion of the region of the person and configured to provide controllable support to said person in at least said portion of said region, and
means for allowing a fluid to enter said at least one bladder, said means configured to controllably receive said fluid so that said at least one bladder controllably inflates and deflates.

2. The apparatus according to claim 1, wherein at least one of said at least one bladder is integrally formed in said garment.

3. The apparatus according to claim 1, comprising a plurality of bladders, each integrally formed in said garment.

4. The apparatus according to claim 1, wherein the garment includes at least one pocket dimensional for receipt of a separate bladder.

5. The apparatus according to claim 1, comprising a plurality of pockets, each dimensional for receipt of a separate bladder.

6. The apparatus according to claim 1, wherein the means for allowing fluid to enter said at least one bladder is a tube coupled to said at least one bladder.

7. The apparatus according to claim 1, further comprising at least one outlet associated with each bladder for release of fluid in said bladder.

8. The apparatus according to claim 7, wherein the at least one outlet is positioned relative to the garment so as to provide said release of fluid in said bladder toward the skin of said person.

9. The apparatus according to claim 1, further comprising a plurality of bladders and a corresponding plurality of support surfaces, and at least one outlet associated with each bladder and corresponding support surface for release of fluid in said bladders.

10. The apparatus according to claim 9, wherein the at least one outlet associated with each bladder and corresponding support surface is positioned so as to provide said release of fluid in said bladder toward the skin of said person.

11. The apparatus of claim 10, wherein said fluid has a therapeutic quality.

12. The apparatus according to claim 1, further comprising:
means for controlling the fluid associated with the means for allowing a fluid to enter said at least one bladder.

13. The apparatus according to claim 12, wherein the means for controlling the fluid comprises:
a fluid pressurizing pump; and
a controller in cooperative engagement with the fluid pressurizing pump, the controller for controlling the inflation and deflation of said at least one bladder.

14. The apparatus according to claim 13, comprising a plurality of bladders and wherein said controller is configured to inflate and deflate said bladders in a predetermined order.

15. The apparatus according to claim 13, comprising a plurality of bladders, wherein the controller is configured to inflate and deflate said bladders in a random manner.

16. The apparatus according to claim 13, wherein the controller is configured to cause inflation and deflation of at least one of the at least one bladder in a random manner.

17. The apparatus according to claim 13, wherein the fluid pressurizing pump is an air pump.

18. The apparatus according to claim 12, wherein the means for allowing a fluid to enter said at least one bladder is a tube extending from said at least one bladder.

19. The apparatus according to claim 18, wherein the means for allowing the fluid to enter said at least one bladder include a quick connect coupler dimensioned for mating with the tube.

20. The apparatus according to claim 12, wherein the means for controlling the fluid delivers a measured volume and pressure of fluid into the at least one bladder, wherein the garment has a diaper-like configuration which positions the at least one bladder beneath said person in a position and orientation so as to reduce pressure exerted on pressure points in order to prevent development of pressure sores and ulceration of skin.

21. The apparatus according to claim 12, comprising means for affixing the garment to said region of the person.

22. The apparatus according to claim 1, comprising means for affixing the garment to said region of the person.

23. The apparatus according to claim 22, wherein said means for affixing comprise lock and loop fasteners.

24. The apparatus according to claim 23, wherein the garment is at least partially formed from a fabric comprising said loops.

25. The apparatus according to claim 22, wherein said means for affixing comprises adhesive tape.

26. The apparatus according to claim 1, comprising at least two bladders in the form of interdigitated fingers.

27. The apparatus according to claim 1, comprising at least three bladders positioned in the garment so as to form a circular pattern.

28. The apparatus according to claim 1, further comprising an absorbent material.

29. The apparatus according to claim 28, wherein said absorbent material is positioned relative to the garment so as to form said at least one corresponding support surface.

30. The apparatus according to claim 1, comprising a plurality of bladders each positioned in the garment so that inflation of any of the bladders does not constrict the garment against the person.

31. The apparatus according to claim 30, wherein the garment comprises flaps for attaching the garment to the person and wherein the plurality of bladders are positioned in the garment away from the flaps.

32. The apparatus according to claim 1, wherein at least one support surface is positioned so as to provide relief of pressure to the person at a desired portion of said region.

33. The apparatus according to claim 1, comprising a plurality of bladders and corresponding plurality of support surfaces so as to impart a turning motion to said region when said bladders are inflated/deflated in a particular pattern.

34. The apparatus according to claim 1, wherein said garment is fabricated from a material including at least one of the following: polyurethane, polyvinylchloride, polyethylene, polypropylene, cotton, biological materials, and biodegradable materials.

35. The apparatus according to claim 1, wherein the garment is fabricated from a reusable material.

36. The apparatus according to claim 1, wherein the garment is fabricated from a disposable material.

37. The apparatus according to claim 1, further comprising means for conditioning the fluid prior to entry in said at least one bladder.

38. The apparatus according to claim 37, wherein said conditioning is at least heating and/or cooling said fluid.

39. The apparatus according to claim 1, wherein the apparatus further comprises a dressing.

40. The apparatus according to claim 39, wherein said dressing is configured to aid in the treatment or prevention of skin ulceration.

41. The apparatus according to claim 1, wherein said at least one bladder and corresponding support surface is configured to provide relief of pressure associated with a bony prominence of said person.

42. A system for preventing or treating ulceration of skin, comprising:
a garment configured for placement about a region of a person, the garment containing a plurality of bladders and a corresponding plurality of support surfaces each configured to be maintained in a substantially fixed relationship relative to a portion of the region of the person and configured to provide controllable support to said person in at least said portion of said region,
means for allowing a fluid to enter each bladder of said plurality of bladders,
a fluid pressurizing pump; and
a controller in cooperative engagement with the fluid pressurizing pump, the controller configured to control the inflation and deflation of each of the plurality of bladders.

43. A method for preventing or treating ulceration of skin, comprising:
placing a garment about a region of a person, the garment containing a plurality of bladders and a corresponding plurality of support surfaces configured to be maintained in a substantially fixed relationship relative to a portion of the region of the person and configured to provide controllable support to said person in at least said portion of said region; and
controllably inflating and deflating each bladder of said plurality of bladders.

44. The method of claim 43, wherein at least some of the fluid exits each of the bladders of said plurality of bladders during deflation thereof directed toward the skin of said person.

45. The method of claim 43, wherein the inflating and deflating of each bladder of said plurality of bladders is in a predetermined order.

46. The method of claim 43, wherein the inflating and deflating of each bladder of said plurality of bladders is performed in a random manner.
